(19) United States
(12) Patent Application Publication
(10) Pub. No.: US 2022/0183873 A1
(43) Pub. Date: Jun. 16, 2022

(54) Cranial positioning device with a neuromuscular effect for the purpose of sea-sickness relief

(71) Applicant: Timothy Dennis McCall, Erie, PA (US)

(72) Inventor: Timothy Dennis McCall, Erie, PA (US)

(21) Appl. No.: 17/118,365

(22) Filed: Dec. 10, 2020

(51) Int. Cl. A61F 5/37; A47G 9/10

(52) U.S. Cl. CPC A61F 5/3707; A47G 9/10

(57) ABSTRACT

This device is a positioning device which positions the head in a way that allows for the relaxation and associated limiting of neural inputs to the lateral sternocleidomastoid muscle. The lateral sternocleidomastoid (to be known as lateral SCM) has been described as a proprioceptive muscle that has a feedback system which may be associated with nautical nausea. This device is shaped much like a pillow and places the user's head in a position which relaxes the lateral SCM muscle and further limits certain neural inputs into said muscle, which subsequently decreases the activity in the cranial feedback system that is associated with this nausea. The end result of this positioning and subsequent lack of neural inputs is an effective relief from nausea associated with typical sea-sickness. The user is required to use the device in a specific manner, which includes 90 seconds of relaxation of the neck and associated resting of the head on the device.

### BACKGROUND

### 1. Field of the Invention

The invention relates generally to cranial positioning device with a neuromuscular effect for the purpose of sea-sickness relief.

### 2. Background

Currently, there may be a number of solutions for cranial positioning. Some of these solutions attempt to position the head with the means to an end, including stretching, strengthening muscles, and relief of cervical vertebral pressure, but these solutions fail to meet the needs of the industry because they do not address the utility of relief of sea sickness, they do target specific muscle group(s) with specific positioning, nor are they utilized with proper technique and timing to this result.

### SUMMARY OF THE INVENTION

It is desirable to have a device which may relieve symptoms associated with sea sickness. Furthermore, it is desirable to have a device that is easy to use in a setting that is most consistent with the development of sea sickness. The disclosed device advantageously fills these needs and addresses the aforementioned deficiencies by providing a position for the head in which, through positional changes in the lateral sternocleidomastoid muscle, is able to relieve symptoms associated with sea sickness.

Disclosed is a sea sickness relief device which is made up of the following components, a supportive wedge shaped pillow that places the head a specific angle as to relieve muscle spasm and neural inputs into the lateral SCM muscle.

The device may also have one or more of the following: a method to change the angle at which the positioning device will operate or a method to change shape in order to allow for different body habitus and head shape to gain benefits from the device.

The disclosed device is unique when compared with other known devices and solutions because it provides: (1) a specific angle with side bending and rotation in which to position in the head and neck targeting the lateral sternocleidomastoid muscle; (2) the utility of relief of muscle spasm related to sea sickness; and (3) the ability to accomplish this on one's own, without the intervention of another person.

The disclosed device is unique in that it is structurally different from other known devices or solutions. More specifically, the device is unique due to the presence of indications on the device as to how to position the user's head and neck, and rigidity to maintain that position.

This disclosure will now provide a more detailed and specific description that will refer to the accompanying drawings. The drawings and specific descriptions of the drawings, as well as any specific or alternative embodiments discussed, are intended to be read in conjunction with the entirety of this disclosure. The Cranial positioning device with a neuromuscular effect for the purpose of sea-sickness relief. may, however, be embodied in many different forms and should not be construed as being limited to the embodiments set forth herein; rather, these embodiments are provided by way of illustration only and so that this disclosure will be thorough, complete and fully convey understanding to those skilled in the art.

### BRIEF DESCRIPTION OF THE DRAWINGS

FIG. 1—Front view of the device

FIG. 2—side view of the device

FIG. 3—top view of the device

FIG. 4—isometric view of the device

### DETAILED DESCRIPTION

The present invention is directed to cranial positioning device with a neuromuscular effect for the purpose of sea-sickness relief

In its most complete version, the device is made up of the following components, a wedge shaped device that has a marked location for the user to place their head upon, with sides right and left as to position the ear over a location on the device that best serves the purpose of sea sickness relief. There are movable components that fold in and out at the base to allow the user to adjust the height and angle of the device as to allow the device to work best with the body habitus of the user. It should further be noted that: The material used in the device is supportive enough to hold the weight of a human head and keep the head in a specific position while allowing the user to relax their musculature completely.

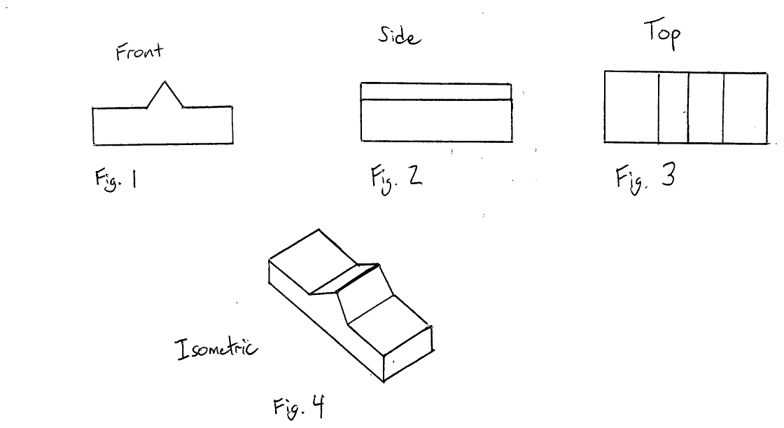

FIG. 1 is a front view of the device.

FIG. 2 is a side view of the device.

FIG. 3 shows a top view of the device, with spots for ear placement marked on the device as to give a better understanding of where the user's head is to be place. The middle of the device has a raised portion that cannot be seen on this view.

FIG. 4 shows an isometric view of the device. It is a raised platform with a wedge protruding from the middle of the platform to help position the user's head. It has spots for ear placement marked on the device as to give a better understanding of where the user's head is to be placed.

Different features, variations and multiple different embodiments have been shown and described with various details. What has been described in this application at times in terms of specific embodiments is done for illustrative purposes only and without the intent to limit or suggest that what has been conceived is only one particular embodiment or specific embodiments. It is to be understood that this disclosure is not limited to any single specific embodiments or enumerated variations. Many modifications, variations and other embodiments will come to mind of those skilled in the art, and which are intended to be and are in fact covered by both this disclosure. It is indeed intended that the scope of this disclosure should be determined by a proper legal interpretation and construction of the disclosure, including equivalents, as understood by those of skill in the art relying upon the complete disclosure present at the time of filing.

1. An apparatus for cranial positioning with a neuromuscular effect for the purpose of sea-sickness relief, the apparatus comprising of a rigid structure that is rigid and large enough to support the head of a person in a specific orientation.
